(19) United States
(12) Patent Application Publication
(10) Pub. No.: US 2006/0281953 A1
(43) Pub. Date: Dec. 14, 2006

(54) Recovery of an active catalyst component from a process stream

(76) Inventors: Hans-Helmut Bechtel, Roetgan (DE); Dietrich Bertram, Aachem (DE); Markus Heinrich Klein, Aachen (DE); Volker Ulrich Weiler, Aachen (DE)

Correspondence Address: PHILIPS INTELLECTUAL PROPERTY & STANDARDS, P.O. BOX 3001, BRIARCLIFF MANOR, NY, 10510, US

(21) Appl. No.: 10/550,044

(22) PCT Filed: Mar. 9, 2004
(86) PCT No.: PCT/IB04/00080

§ 371 Date: Sep. 21, 2005

(51) Int. Cl. B01J 20/34; C07C 45/50

(52) U.S. Cl. 568/397; 502/29

(57) ABSTRACT

A process for recovering an active catalyst component from a process stream containing, in addition to dissolved active catalyst component, also at least aldols, acetals and/or esters, includes admixing an alcohol component comprising at least one C1 to C10 alcohol, with the process stream to form solid active catalyst component. The solid active catalyst component is recovered from a residual alcohol-rich phase.

THIS INVENTION relates to the recovery of an active catalyst component from a process stream. More particularly, the invention relates to a process for recovering an active catalyst component from a process stream, and to a hydroformylation process.

According to a first aspect of the invention, there is provided a process for recovering an active catalyst component from a process stream containing, in addition to dissolved active catalyst component, also at least aldols, acetals and/or esters, which process includes
admixing an alcohol component comprising at least one C₁ to C₁₀ alcohol, with the process stream to form solid active catalyst component; and
recovering the solid active catalyst component from a residual alcohol-rich phase.

The process stream may, in particular, be a hydroformylation process heavies purge stream as hereinafter described, while the active catalyst component may comprise a metal/carbon monoxide/ligand complex and, optionally, free ligand, as also hereinafter described.

The hydroformylation process (or Oxo process) is a catalytic process for the conversion, at elevated temperature and pressure, of an olefin into an aldehyde and/or alcohol product having one carbon more than the starting olefin by the addition of one molecule each of hydrogen and carbon monoxide to the carbon-carbon double bond of the olefin, thereby adding a carbonyl or carbinol group to an olefinically unsaturated carbon atom and saturating the olefin bond.

The hydroformylation catalyst is selected according to the particular oxygenated products which are required from a particular olefinic feedstock.

The catalyst used is, however, typically a homogeneous (liquid phase) catalyst, and may comprise a mixture or complex of a metal, for example, cobalt (Co), rhodium (Rd), ruthenium (Ru) or palladium (Pd); carbon monoxide; and a phosphine and/or phosphate ligand. Examples of such catalysts are triphenyl phosphine ligands used with rhodium, and alkyl phosphine ligands used with cobalt. Specific examples of the latter are trialkyl phosphines and bicyclic tertiary phosphines such as 9-phosphabicyclo [3.3.1] nonane and 9-phosphabicyclo [4.2.1] nonane represented by formulas (I) and (II) respectively:

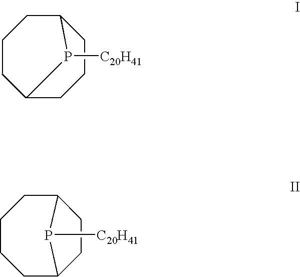

The ligands I and II are available commercially, as a mixture, under the collective chemical name eicosyl phoban (‘EP’).

The catalyst mixture or complex is preferably used as an admixture with an aromatic, olefinic and/or paraffinic organic solvent. The organic solvent may thus be a common solvent such as toluene, or a long chain paraffin. However, the olefinic feedstock of the hydroformylation process in which the catalyst is to be used, can also be used as the organic solvent, if desired.

Thus, in the hydroformylation process, an olefin-containing feedstock is reacted, in a reaction zone and in the presence of the catalyst, and at elevated temperature and pressure, with carbon monoxide and hydrogen, with olefins in the feedstock thus reacting with carbon monoxide and hydrogen to form aldehydes and/or alcohols; a reaction mixture comprising unreacted feedstock, alcohols, aldehydes, catalyst residue and, optionally, unreacted gaseous reactants, is withdrawn from the reaction zone and passed to a separation zone to separate liquid and gaseous phases; the liquid phase passes to a distillation zone where the alcohols, aldehydes and unreacted feedstock are separated from the catalyst residue; the catalyst residue is withdrawn as a bottoms component and recycled to the reaction zone; and the alcohols, aldehydes and unreacted feedstock are withdrawn from the distillation zone as an overheads component.

The catalyst residue thus contains the active catalyst component, which may be the metal/carbon monoxide/ligand complex, and, optionally, free ligand.

During the hydroformylation reaction, heavies or bottoms high boiling by-products are formed as a result of reactions between the products. Specifically, aldols, acetals and esters are formed during the hydroformylation reaction. More specifically, aldols are formed as a result of aldol condensation between two aldehyde molecules. Esters are formed as a result of the reaction between acids (resulting from the metal in the catalyst) and alcohols. Finally, acetals are formed as a result of the reaction between alcohols and aldehyde molecules formed during the reaction.

Since these products are not removed in the distillation zone overheads component, they are recycled back to the reaction zone as part of the bottoms component. With continuous recycle, there is thus a continuous build-up of the heavies in the reactor. Over time these heavies will build up to unacceptable levels if not removed. Typically, a constant level of 25% (mass basis) heavies content in the reaction zone is required. In order to keep the heavies level constant, they need to be removed at the same rate as that in which they are formed. This is accomplished by removing (purging) part of the bottoms component or recycle stream, eg by means of a pump, as the heavies purge stream hereinbefore referred to.

The continuous purging of heavies also leads to a loss of expensive catalyst, ie metal/carbon monoxide/ligand complex, and, optionally, free ligand, from the hydroformylation cycle. Although the ligand and catalyst lost during purging can be replenished, the continuous loss and lack of recovery of the catalyst and ligand can have severe implication on the process economics, especially if an expensive ligand and/or catalyst source is used. Thus, such loss, on a commercial scale operation, represents a high economic penalty which can make the process prohibitively expensive. It is for this reason that a cost effective quantitative method to recover the catalyst and/or optionally free ligand from the heavies is highly desirable. The present invention provides such a cost-effective quantitative recovery method.

In addition, the loss of catalyst and ligand may have severe environmental implication in considering the poisonous nature of the catalyst metal (eg cobalt) and phosphorus based ligand.

An added advantage of the process of the invention is that the inactive catalyst component, namely ligand phosphine oxide, and which also forms part of the catalyst residue, remains in the residual alcohol-rich phase, thus circumventing the build-up of the oxide in the reaction zone over time. The heavies also remain in the residual alcohol-rich phase. The heavies can be recovered from the residual alcohol-rich phase, and the recovered heavies could then, in the absence of catalyst and free ligand, be cracked back to the product components (alcohols and aldehydes), adding another economic benefit to the process.

According to a second aspect of the invention, there is provided a hydroformylation process, which includes
reacting, in a reaction zone and in the presence of a hydroformylation catalyst, and at elevated temperature and pressure, an olefin-containing feedstock with carbon monoxide and hydrogen, to form aldehydes and/or alcohols;
withdrawing a reaction mixture comprising the alcohols, the aldehydes, unreacted feedstock, catalyst residue, heavies and, optionally, unreacted gaseous reactants, from the reaction zone;
in a separation zone, separating a gaseous phase from a liquid phase comprising the aldehydes, alcohols, unreacted feedstock, the heavies and the catalyst residue;
in a distillation zone, subjecting the liquid phase to distillation;
withdrawing from the distillation zone, as an overheads component, the alcohols, aldehydes and unreacted feedstock;
withdrawing from the distillation zone, as a bottoms component, the heavies and the catalyst residue, which comprises an active catalyst component and at least some of which is in solution;
admixing an alcohol component comprising at least one C₁ to C₁₀ alcohol, with at least a portion of the bottoms component to form solid active catalyst component; and
recovering the solid active catalyst component from a residual alcohol-rich phase.

The hydroformylation catalyst may be as hereinbefore described. Thus, the active catalyst component may comprise a metal/carbon monoxide/ligand complex and, optionally, free ligand, as also hereinbefore described.

The process may include recycling a portion of the bottoms component to the reaction zone, with the portion thereof that is admixed with the alcohol component thus constituting a heavies purge stream that is withdrawn.

While the active catalyst component can, in the heavies purge stream, be wholly in solution, the Applicant has found that in practice the active catalyst component will normally only be totally dissolved at high temperatures, eg at about 40° C. or higher. Usually, however, the heavies purge stream will be at a lower temperature, eg at room temperature, when the alcohol component is added to it, so that some active catalyst component solids formation may already have taken place. However, the quantity of solid active catalyst component thus transformed into solid form is significantly less (and may even be negligible) than that resulting from the admixing of the alcohol component with the heavies purge stream.

The process of the invention is characterized thereby that no dilution of the bottoms component, at least prior to the withdrawal of the heavies purge stream therefrom, or of the heavies purge stream, with a saturated or unsaturated aliphatic hydrocarbon having 3 to 20 carbon atoms or with an aromatic or hydrocarbyl-substituted aromatic hydrocarbon having from 6 to 22 carbon atoms, takes place.

The addition of the alcohol component comprising the C₁ to C₁₀ alcohol, ie an alcohol containing from 1 to 10 carbon atoms, to the heavies purge stream thus leads to the selective solidification of the metal/carbon monoxide/ligand complex and, optionally, free phosphorus ligand. As mentioned hereinbefore, the catalyst residue will normally contain, in addition to the active catalyst component, also inactive catalyst components such as phosphine ligand oxides. These phosphine ligand oxides and heavies will not solidify, but will remain in the alcohol-rich phase.

The alcohol component may comprise at least one C₁ to C₁₀ monohydric alcohol and/or at least one C₁ to C₁₀ dihydric alcohol, ie an alcohol containing two hydroxyl groups.

The alcohol of the alcohol component may be in concentrated or undiluted form.

When the alcohol component comprises or consists of a monohydric alcohol, the monohydric alcohol preferably has a C₁ to C₈ carbon chain; more preferably, it is then a concentrated or undiluted primary alcohol having 1 to 3 carbon atoms. Most preferably, concentrated or undiluted methanol or ethanol is then used as the alcohol component.

When the alcohol component comprises or consists of a dihydric alcohol, the dihydric alcohol preferably has a C₂ to C₈ carbon chain. For example, undiluted or concentrated ethylene glycol or propylene glycol can then be used as the alcohol component.

The Applicant has also found that while a single monohydric or dihydric alcohol can be used for efficient ligand and/or catalyst component recovery, even more efficient recoveries can, in certain circumstances, be achieved by using a mixture of two or more undiluted or concentrated alcohols as the alcohol component. For example, an undiluted dihydric alcohol such as ethylene glycol or propylene glycol can effectively be used in admixture with at least one other undiluted dihydric alcohol and/or with at least one undiluted monohydric alcohol. The alcohol mixture will then naturally be admixed with the process stream or with the bottoms component, as the case may be.

It has thus been discovered that the addition of a C₁ to C₁₀ alcohol, and more specifically a C₁ to C₅ alcohol, such as a C₁ to C₃ monohydric alcohol and/or a C₂/C₃ dihydric alcohol, to the heavies purge stream leads to the recovery of an active catalyst component, viz metal/carbon monoxide/ligand complex, and, optionally, free ligand.

Although the alcohol component can be at room temperature, it has unexpectedly been found that the use of chilled alcohol, and in particular chilled methanol, results in particularly effective solidification of the active catalyst component. The alcohol component, on admixture thereof with the process stream or with the bottoms component, is thus preferably at a temperature below room temperature, more preferably at a temperature below 0° C., eg at about −5° C. Naturally, the alcohol must be in liquid form, and it will thus be at a temperature above its freezing temperature.

The formation of the solid active catalyst component typically involves precipitation or crystallization, ie the active catalyst component is allowed to precipitate or crystallize. When precipitation occurs, the active catalyst component, on solidifying, yields amorphous solids whose shape and size are not well defined. When crystallization occurs, the active catalyst component, on solidifying, yields solid particles that are of well-defined shape and size or that have the form and structure of crystals.

The process may include subjecting the process stream or the bottoms component, after admixture of the alcohol component therewith, to centrifugation.

The recovery of the solid active catalyst component from the residual alcohol-rich phase may be effected by means of any suitable solids recovery method including, but not limited to, one or more of filtration, centrifugation, clarification or sedimentation, hydrocyclonic action, elutriation, fractional crystallization, concentration, flotation, expression, magnetic separation, and electrostatic separation.

The process may include recycling the recovered active catalyst component to the reaction zone.

The alcohol-rich phase can also be processed further, if desired, eg to recover the alcohol for re-use.

The reaction temperature may be from 100° C. to 300° C., typically from 150° C. to 200° C.

The reaction pressure may be at least 20 bar (150 psi), preferably between 50 bar (750 psi) and 100 bar (1500 psi), typically about 85 bar (1232 psi).

The hydroformylation reaction zone may be provided by a reactor capable of handling a homogenously catalysed chemical transformation, such as a continuous stirred tank reactor (‘CSTR’), bubble column, or the like.

The olefin-containing feedstock may, in particular, be a C₂ to C₂₀ Fischer-Tropsch derived olefin stream. Thus, the olefin-containing feedstock may be that obtained by subjecting a synthesis gas comprising carbon monoxide and hydrogen to Fischer-Tropsch reaction conditions in the presence of an iron-based, a cobalt-based or an iron/cobalt-based Fischer-Tropsch catalyst, with the resultant olefinic product then constituting the olefin-containing feedstock of the process of the invention, or a component thereof constituting the olefin-containing feedstock of the process of the invention.

In other words, the olefinic product from the Fischer-Tropsch reaction can, if necessary, be worked up to remove unwanted components therefrom and/or to separate a particular olefinic component therefrom, with said particular olefinic component then constituting the olefin-containing feedstock of the process of the invention.

The invention will now be described in more detail, with reference to the accompanying drawings.

In the drawings,

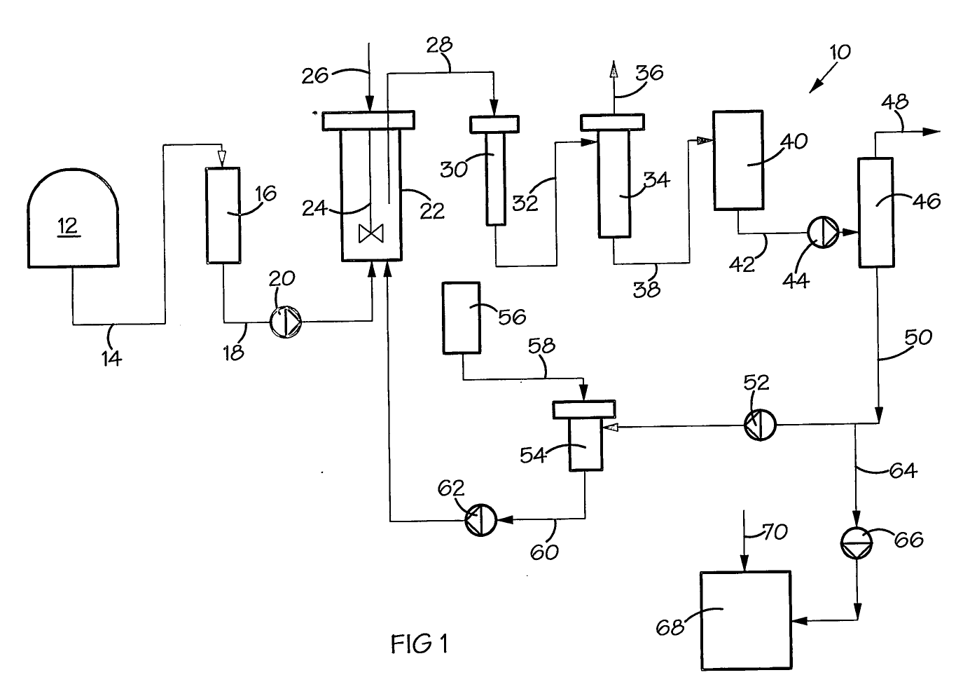

FIG. 1 shows a flow diagram of a hydroformylation process demonstration unit (‘PDU’);

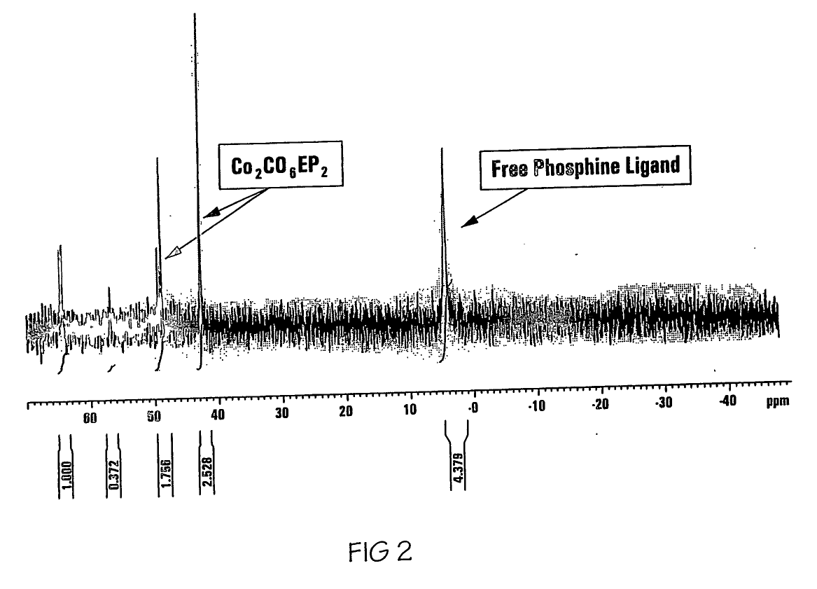

FIG. 2 shows a ³¹P NMR spectrum of the purge derived precipitate obtained in Example 1;

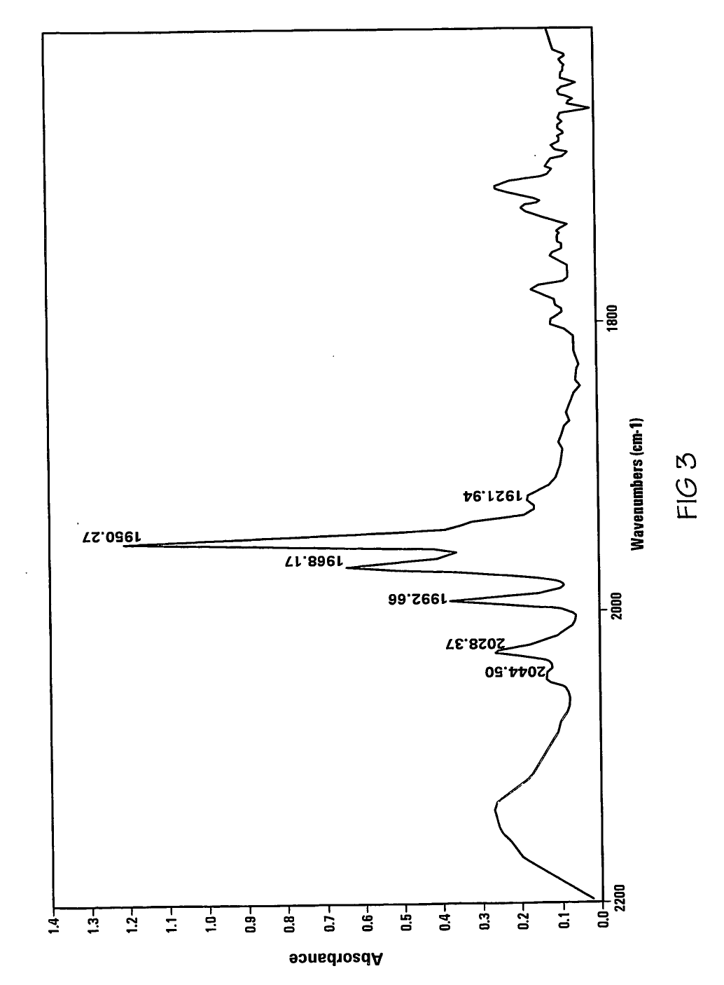

FIG. 3 shows an IR spectrum of the purge derived precipitate obtained in Example 1.

In the drawings, reference numeral 10 generally indicates a flow diagram of a hydroformylation or oxo process demonstration unit (‘PDU’). The PDU is used to test the continuous operation of the oxo process.

The PDU 10 includes an olefin-containing feedstock container 12 connected, by means of a feed line 14, to a feed pot 16. A flow line 18, fitted with a feed pump 20, leads from the feed pot 16 to a hydroformylation reactor 22.

The reactor 22 is fitted with a stirrer 24, and a syngas feed line 26 also leads into the reactor 22. A reaction mixture withdrawal line 28 leads from the reactor 22 to a knockout pot 30, with a flow line 32 leading from the knockout pot 30 to a level pot 34. A gas phase withdrawal line 36 leads from the level pot 34 as does a liquid phase withdrawal line 38. The line 38 leads to a feed pot 40, with a flow line 42, fitted with a feed pump 44, leading from the feed pot 40 into a short path distillation (‘SPD’) unit 46. An overheads line 48, for withdrawing an overheads component, leads from the unit 46 as does a bottoms component withdrawal line 50. The line 50 is fitted with a discharge pump 52 and leads to a purge pot 54.

The PDU 10 includes a catalyst feed pot 56, with a flow line 58 leading from the feed pot 56 to the purge pot 54. A catalyst recycle line 60, fitted with a recycle pump 62, leads from the purge pot 54 back to the reactor 22.

In use, a Fischer-Tropsch derived olefin-containing feedstock containing a range of alpha alkenes, is fed to the reactor 22 from the feed pot 16. Syngas, containing carbon monoxide and hydrogen, is also fed into the reactor 22 by means of the flow line 26. The reactor 22 contains a homogeneous (liquid phase) catalyst comprising a mixture or complex of cobalt, carbon monoxide and phosphine ligand (EP) mixed with toluene as an aromatic organic solvent.

The alkenes react with the carbon monoxide and hydrogen to form aldehydes and alcohols, in accordance with the so-called oxo reaction. The hydroformylation reaction typically takes place at 85 bar and 170° C.

A reaction mixture comprising aldehydes, alcohols, unreacted feedstock, heavies, catalyst residue and unreacted gaseous reactants, leaves the reactor 22 along the flow line 23 and passes into the knockout pot 30 and then into the level pot 34 where liquid and gas phases are separated. The gas phase is withdrawn along the flow line 36, while the liquid phase, comprising unreacted feedstock, aldehydes, alcohols, heavies and catalyst residue, is withdrawn along the flow line 38. The liquid phase is depressurized across a level control valve in the level pot 34 and then passes to the feed pot 40. From the feed pot 40, the liquid phase is pumped, by means of the line 42 and pump 44, to the SPD unit 46, where the alcohols, aldehydes and unreacted olefin-containing feedstock are separated from the heavies and the catalyst residue, and withdrawn as an overheads component, along the flow line 48. The heavies and catalyst residue are withdrawn, as a bottoms component, and pass along the flow line 50 to the purge pot 54, where makeup catalyst is added thereto from the catalyst feed pot 56. The bottoms component then passes, from the purge pot 54, back to the reactor 22, by means of the line 60 and the pump 62. The catalyst residue comprises active catalyst components, namely cobalt/carbon monoxide/ligand complex and, optionally, ligand on its own, together with inactive catalyst components such as ligand oxide.

The PDU 10 is thus a mini pilot plant or rig which is used to test a hydroformylation process on a large scale, before upgrading to pilot plant scale.

During the hydroformylation reaction, heavies (high boiling by-products) are formed as hereinbefore described. These heavies are not removed in the SPD unit 46, and are thus recycled back to the reactor 22 together with the catalyst residue. Over time, these heavies build up to unacceptable levels, if not removed. Typically, a constant level of 25% (mass basis) heavies content in the reactor 22 is required. In order to keep the heavies level in the reactor 22 constant, they need to be removed at the same rate at which they are formed. This is accomplished by removing (purging) part of the recycle stream, along a purge line 64 fitted with a pump 66. The line 64 leads into a container 68 with a nitrogen line 70 also leading into the container 68. The purge stream is thus collected in the container 68 that is blanketed with nitrogen to prevent the purge stream from oxidizing. The purge stream can be recovered from the container 68 for further investigation.

The continuous purging of heavies also leads to a loss of expensive catalyst complex and/or ligand from the hydroformylation process, as hereinbefore described. Thus, although the ligand and catalyst lost during the purging can be replenished, the continuous loss and lack of recovery thereof can have severe implication on process economics. For example, an 8 kilo ton/annum purge stream from a commercial hydroformylation plant would contain 422 ton/annum of phosphine which could be lost if not recovered efficiently.

The catalyst addition system comprising the catalyst feed pot 56 and the line 58 was installed, in the PDU unit 10, primarily to replace catalyst that is purged via the purge line 64. However, the catalyst addition system, since it is a closed system, also prevents any oxidation of the catalyst. The catalyst addition system can also be used at start-up to introduce catalyst into the process.

The purge stream withdrawn along the line 64 is thus a mixture of heavies, dissolved active cobal/tcarbon monoxide/ligand complex or catalyst, and, optionally, free phosphine ligand, and inactive phosphine ligand oxide.

This purge stream was treated with alcohol in accordance with the invention, as described in Examples 1 and 5 hereunder.

### EXAMPLE 1

Typically, 50 ml of the purge stream from the PDU unit 10 was treated, in a 500 ml separating funnel, with 250 ml to 300 ml concentrated or undiluted methanol (100%) chilled to about −5° C. After vigorous shaking, the solution was allowed to separate out over a period of 10 to 30 minutes. During this period, a golden yellow solid particulate component formed by means of precipitation or crystallization, and settled out of the bottom of the flask. This solid particulate component was separated from the residual methanol phase and subjected to careful filtration under argon, to recover an active catalyst component, namely cobalt/carbon monoxide/phosphine ligand complex. The active catalyst component was dried at room temperature under a flow of argon gas. The recovered catalyst component was then weighed and analyzed by ³¹P NMR and infrared (‘IR’) spectroscopy. The methanol phase was evaporated off to recover the methanol, leaving a residue containing heavies and phosphine ligand oxide.

FIG. 2 is the ³¹P NMR spectrum for the recovered catalyst precipitate after drying, while FIG. 3 is an infrared (IR) spectrum of the recovered precipitate. In FIG. 2, the peak at 1.88 and −38 pm is for free phosphine ligand (EP). In FIG. 3, the peak assignments are as follows: 1921, 1950 cm⁻¹: Co₂(CO)₆EP₂, 1968cm⁻¹: HCo(CO)₃EP, 2028 cm⁻¹: Co₂(CO)₇EP.

Re-use of the recovered solid particulate catalyst component, ie the recovered cobalt/carbon monoxide/phosphine ligand complex, was demonstrated in Examples 2 to 4. In Examples 2 to 4, the reactor that was used in each case was a Parr autoclave reactor, ie a batch reactor.

### EXAMPLE 2

In a typical experiment, 1.5 g of the solid particulate catalyst component of Example 1 was introduced into the 100 ml autoclave to yield a cobalt concentration of 1000 ppm. 50 ml of the olefin feedstock, namely depentemizer overheads (DPO), was introduced. DPO is a Fischer-Tropsch derived feed which contain C₅ olefins including 70% 1-pentene. The reaction mixture was then heated to 170° C. After this temperature was reached, the mixture was pressurized to 75 bar with syngas having a 2:1 H₂:CO molar ratio. The reaction mixture was then left to react for 3 hours with syngas fed on demand. After the allotted time, the mixture was cooled to room temperature, and thereafter a sample of the reaction mixture was obtained and analyzed by GC and GC-MS.

Results:

From Catalyst re-use: % 1-pentene conversion: 97.3%, C₆ alcohol linearity=85.6%: n:i=15.8.

From PDU run (Example 1): % 1-pentene conversion: 98.2%, C₆ alcohol linearity=87.9%: n:i=20.1.

### EXAMPLE 3

In a typical experiment, 1.5 g of the solid particulate catalyst component of Example 1 was introduced into the 100 ml autoclave. 50 ml of olefin feedstock, namely 100% 1-pentene, was introduced into the autoclave. The solution was then heated to 170° C. After this temperature was reached, the mixture was pressurized to 75 bar with syngas having a 2:1 H₂:CO molar ratio. The reaction mixture was then left to react for 3 hours with syngas fed on demand. After the allotted time, the mixture was cooled, and thereafter reacted, sampled and analyzed by GC and GC-MS.

Results:

From Catalyst re-use (precipitate): % 1-pentene conversion: 97.1%, C₆ alcohol linearity=93.4%: n:i=17.6.

*From freshly prepared catalyst run: % 1-pentene conversion: 95.4%, C₆ alcohol linearity=95.4%: n:i=19.8.
* Cobalt Concentration=1000 ppm. Ligand:Metal ratio=3:1. This run was then also conducted in the Parr autoclave; all conditions were identical to those using the recovered catalyst precipitate except that fresh catalyst was used; the ligand to metal ratios in the recovered catalyst component experiments were similar to those in the fresh catalyst experiment.

### EXAMPLE 4

In a typical experiment, 1.5 g of the solid particulate catalyst component of Example 1 was introduced into the 100 ml autoclave. 50 ml of olefin feedstock, namely C₇ olefin, 1-heptene, was introduced into the autoclave. The solution was then heated to 170° C. After this temperature was reached, the mixture was pressurized to 75 bar with syngas having a 2:1 H₂:CO molar ratio. The reaction mixture was then left to react for 3 hours with syngas fed on demand. After the allotted time, the mixture was cooled, and thereafter reacted, sampled and analyzed by GC and GC-MS.

Results:

From Catalyst re-use: % 1-heptene conversion: 98.4%, C₈ alcohol linearity=87.9%: n:i=18.5.

*From freshly prepared catalyst run: % 1-heptene conversion: 97.5%, C₈ alcohol linearity=86.6%: n:i=18.6
* Cobalt Concentration=1000 ppm. Ligand:Metal ratio=3:1. This run was then also conducted in the Parr autoclave; all conditions were identical to those using the recovered catalyst precipitate except that fresh catalyst was used; the ligand to metal ratios in the recovered catalyst component experiment were similar to those in the fresh catalyst experiment.

### EXAMPLE 5

About 50 ml of the purge stream from the PDU unit 10 was mixed with 150 ml of concentrated or undiluted methanol (100%) which was at room temperature (25° C.). The dissolved active catalyst component was transformed into solid form, and the resultant mixture was centrifuged at 10000 rpm for one hour. The solid active catalyst component was then recovered by means of filtration. The recovered solid particulate active catalyst component was analyzed for cobalt and phosphorus content using Inductive Coupled Plasma Spectroscopy (ICP). The results obtained showed that 50% of the total phosphorus and cobalt content was in the solid phase, ie in the insoluble component. This ICP analysis thus shows a high recovery of the active catalyst component, since the balance of the phosphorus is in the non-recovered inactive phosphine ligand oxide. Cobalt (in catalyst form) recovery is at least 70-75%.

It has thus been shown that a precipitated or crystallized catalyst, ie active catalyst components, recovered in accordance with the invention, can be re-used in batch autoclave runs. It has also been shown that the recovered catalyst can be used in a continuous demonstration unit process without any marked or significant decrease in catalyst activity and/or product quality and/or selectivity.

1. A process for recovering an active catalyst component from a process stream containing, in addition to dissolved active catalyst component, also at least aldols, acetals and/or esters, which process includes
admixing an alcohol component comprising at least one C₁ to C₁₀ alcohol, with the process stream to form solid active catalyst component; and
recovering the solid active catalyst component from a residual alcohol-rich phase.

2. A process according to claim 1, wherein the process stream is a hydroformylation process heavies purge stream, while the active catalyst component comprises a metal/carbon monoxide/ligand complex and, optionally, free ligand.

3. A process according to claim 1 or claim 2, wherein the alcohol component comprises at least one C₁ to C₁₀ monohydric alcohol and/or at least one C₁ to C₁₀ dihydric alcohol.

4. A process according to claim 3, wherein the alcohol of the alcohol component is in concentrated or undiluted form.

5. A process according to claim 4, wherein the alcohol component comprises a concentrated monohydric alcohol having 1 to 3 carbon atoms.

6. A process according to claim 5, wherein the monohydric alcohol of the alcohol component is concentrated methanol or concentrated ethanol.

7. A process according to claim 4, wherein the alcohol component comprises a concentrated dihydric alcohol having 2 to 8 carbon atoms.

8. A process according to claim 7, wherein the dihydric alcohol component of the alcohol component is concentrated ethylene glycol or concentrated propylene glycol.

9. A process according to claim 4, wherein the alcohol component comprises a mixture of at least two concentrated C₁ to C₁₀ alcohols.

10. A process according to any one of claims 1 to 9 inclusive, wherein the alcohol component, on admixture thereof with the process stream, is at a temperature below room temperature and above its freezing temperature.

11. A process according to claim 10, wherein the alcohol component is at a temperature below 0° C.

12. A process according to any one of claims 1 to 11 inclusive, wherein the formation of the solid active catalyst component is by means of precipitation or crystallization.

13. A process according to any one of claims 1 to 12 inclusive, which includes subjecting the process stream, after admixture of the alcohol component therewith, to centrifugation.

14. A hydroformylation process, which includes
reacting, in a reaction zone and in the presence of a hydroformylation catalyst, and at elevated temperature and pressure, an olefin-containing feedstock with carbon monoxide and hydrogen, to form aldehydes and/or alcohols;
withdrawing a reaction mixture comprising the alcohols, the aldehydes, unreacted feedstock, catalyst residue, heavies and, optionally, unreacted gaseous reactants, from the reaction zone;
in a separation zone, separating a gaseous phase from a liquid phase comprising the aldehydes, alcohols, unreacted feedstock, the heavies and the catalyst residue;
in a distillation zone, subjecting the liquid phase to distillation;
withdrawing from the distillation zone, as an overheads component, the alcohols, aldehydes and unreacted feedstock;
withdrawing from the distillation zone, as a bottoms component, the heavies and the catalyst residue, which comprises an active catalyst component and at least some of which is in solution;
admixing an alcohol component comprising at least one C₁ to C₁₀ alcohol, with at least a portion of the bottoms component to form solid catalyst component; and
recovering the solid active catalyst component from a residual alcohol-rich phase.

15. A process according to claim 14, wherein the active catalyst component comprises a metal/carbon monoxide/ligand complex and, optionally, free ligand.

16. A process according to claim 14 or claim 15, which includes recycling a portion of the bottoms component to the reaction zone, with the portion thereof that is admixed with the alcohol component thus constituting a heavies purge stream that is withdrawn.

17. A process according to claim 16, wherein no dilution of the bottoms component, at least prior to the withdrawal of the heavies purge stream therefrom, or of the heavies purge stream, with a saturated or unsaturated aliphatic hydrocarbon having 3 to 20 carbon atoms or with an aromatic or hydrocarbyl-substituted aromatic hydrocarbon having from 6 to 22 carbon atoms, takes place.

18. A process according to any one of claims 14 to 17 inclusive, wherein the alcohol component comprises at least one C₁ to C₁₀ monohydric alcohol and/or at least one C₁ to C₁₀ dihydric alcohol.

19. A process according to claim 18, wherein the alcohol of the alcohol component is in concentrated or undiluted form.

20. A process according to claim 19, wherein the alcohol component comprises a concentrated monohydric alcohol having 1 to 3 carbon atoms.

21. A process according to claim 20, wherein the monohydric alcohol of the alcohol component is concentrated methanol or concentrated ethanol.

22. A process according to claim 19, wherein the alcohol component comprises a concentrated dihydric alcohol having 2 to 8 carbon atoms.

23. A process according to claim 22, wherein the dihydric alcohol component of the alcohol component is concentrated ethylene glycol or concentrated propylene glycol.

24. A process accoding to claim 19, wherein the alcohol component comprises a mixture of at least two concentrated C₁ to C₁₀ alcohols.

25. A process according to any one of claims 14 to 24 inclusive, wherein the alcohol component, on admixture thereof with the bottoms component, is at a temperature below room temperature and above its freezing temperature.

26. A process according to claim 25, wherein the alcohol component is at a temperature below 0° C.

27. A process according to any one of claims 14 to 26 inclusive, wherein the formation of the solid active catalyst component is by means of precipitation or crystallization.

28. A process according to any one of claims 14 to 27 inclusive, which includes subjecting the bottoms component, after admixture of the alcohol component therewith, to centrifugation.
